SEQUENCE LISTING (8 sequences)

SEQ ID NO: 1 - DNA, 20 bp, Artificial Sequence
  A primer which was synthesized in a laboratory
cagctcagtaacagtccgcc

SEQ ID NO: 2 - DNA, 20 bp, Artificial Sequence
  A primer which was synthesized in a laboratory
tcactattggcaacgagcgg

SEQ ID NO: 3 - DNA, 20 bp, Artificial Sequence
  A primer which was synthesized in a laboratory
cctggagatcccgttgcttc

SEQ ID NO: 4 - DNA, 20 bp, Artificial Sequence
  A primer which was synthesized in a laboratory
tcccggtcagtctttgcttc

SEQ ID NO: 5 - DNA, 20 bp, Artificial Sequence
  A primer which was synthesized in a laboratory
aaatcactctttaagaccag

SEQ ID NO: 6 - DNA, 20 bp, Artificial Sequence
  A primer which was synthesized in a laboratory
ttattgaatagcagtgacag

SEQ ID NO: 7 - DNA, 20 bp, Artificial Sequence
  A primer which was synthesized in a laboratory
atctctctgtaccctctgca

SEQ ID NO: 8 - DNA, 20 bp, Artificial Sequence
  A primer which was synthesized in a laboratory
cctctcttggtgtccataca

(19) United States
(12) Patent Application Publication
(10) Pub. No.: US 2019/0070129 A1
(43) Pub. Date: Mar. 7, 2019

(54) COMPOSITION COMPRISING PANDURATIN OR FINGERROOT (BOESENBERGIA PANDURATA) EXTRACT FOR TREATING, PREVENTING, OR AMELIORATING BONE LOSS DISEASE

(71) Applicant: Newtree Co., Ltd., Seongnam-si (KR)

(72) Inventors: Jae Kwan Hwang, Seoul (KR); Si Yeon Kim, Goyang-si (KR); Mi Bo Kim, Seogwipo-si (KR)

(21) Appl. No.: 16/115,018

(22) Filed: Aug. 28, 2018

Related U.S. Application Data
(63) Continuation-in-part of application No. PCT/KR2017/002321, filed on Mar. 3, 2017.

(30) Foreign Application Priority Data
Mar. 4, 2016 (KR) 10-2016-0026317

(51) Int. Cl. A61K 31/12; A61P 19/08; A61K 36/906; A61K 9/00; A23L 33/105; A23L 2/52; A23G 4/06; A23G 3/48; A21D 2/36

(52) U.S. Cl. CPC A61K 31/12; A61P 19/08; A61K 36/906; A61K 9/0056; A23L 33/105; A23V 2002/00; A23G 4/068; A23G 3/48; A21D 2/36; A61K 2236/333; A23L 2/52

(57) ABSTRACT

The present invention relates to use of a composition comprising a panduratin derivative or a boesenbergia pandurata extract as an effective ingredient, which suppresses the differentiation of osteoclasts to exhibit the effect of treating, preventing or ameliorating bone loss diseases and which can be used safely as a natural material without side effects, thus finding effective applications in drugs, quasi-drugs, or foods.

### CROSS-REFERENCE TO RELATED APPLICATIONS

This application is a continuation-in-part of PCT patent application Ser. No. PCT/KR2017/002321, filed on Mar. 3, 2017, which claims a priority from Korean Patent Application No. 10-2016-0026317, filed on Mar. 4, 2016, the disclosures of both applications are herein incorporated by reference in its entirety.

### TECHNICAL FIELD

The present invention relates to a composition containing a panduratin derivative or a Boesenbergia pandurata extract for preventing, ameliorating, or treating a bone disease.

### BACKGROUND

For normal bone functions, a bone remodeling process through homeostasis of bone resorption by osteoclasts and bone formation by osteoblasts is needed. However, the excessive activity of osteoclasts and the activity degradation of osteoblasts cause imbalance in the remodeling process, resulting in bone loss diseases (skeletal diseases) such as osteoporosis.

Osteoporosis is the most frequent bone loss disease among bone-related diseases, such as multiple myeloma and osteoarthritis, and is generally characterized by an increase in fracture and a decrease in bone strength (Nat. Rev. Endocrinol. 8: 212-227, 2011; J. Dent. Res. 91: 736-744, 2012).

In the oral cavity, a rapid increase in osteoclast activity of the alveolar bone results in bone resorption, causing a loss of alveolar bone in the periodontal tissue, resulting in an alveolar bone loss disease accompanied by tooth loss (J. Immunol. Res. 2015: 1-10, 2015). In order to overcome such an imbalance, a method of inhibiting excessive activity of osteoclasts, promoting osteoblast activity, or inhibiting osteoclast activity and promoting osteoclast activity is generally used (Nat. Rev. Rheumatol. 7: 631-638, 2011).

Osteoclasts are formed in hematopoietic stem cells, and the differentiated osteoclasts decompose mineralized bones, and thus play an important role in the growth of body skeleton and the maintenance of homeostasis. The differentiation of osteoclasts is regulated by receptors, such as receptor activator of nuclear factor kappa-B ligand (RANKL) as a tumor necrosis factor (TNF) ligand, receptor activator of nuclear factor kappa-B (RANK), and osteoprotegerin (OPG). RANKL is secreted from cells, such as osteoblasts or human gingival fibroblasts, and attached to RANK expressed in osteoclasts or osteoclast precursor cells. Such an attachment signal increases the activity of nuclear factor of activated T-cells, cytoplasmic 1 (NFATc1), which is a major transcription factor, and thus, promotes the synthesis of an enzyme, such as tartrate-resistant acid phosphatase (TRAP), and a calcitonin receptor that is a specific biomarker of osteoclast differentiation, which plays a major role in bone degradation and resorption. OPG as a decoy receptor is attached to RANKL to prevent the direct attachment of RANKL to RANK. Therefore, the relative concentrations of RANKL and OPG play an important role in maintaining skeletal homeostasis (J. Immunol. Res. 2015: 1-10, 2015).

Representative osteoporosis medicines that have been developed so far are mainly bisphosphonate preparations that inhibit bone loss by weakening osteoclast functions. However, these chemical synthetic products have a disadvantage of causing sides effects, such as gastrointestinal disorder, kidney toxicity, musculoskeletal pain, and jaw necrosis. Therefore, the prevention and treatment of osteoporosis or alveolar bone loss based on natural materials with characteristics of few side effects and high safety are very important research subjects (Datamonitor Research Reports, 2007; Korean J. Fam. Pract. 3: 16-24, 2013).

Boesenbergia pandurata (Boesenbergia rotunda as the same name) is a Zingiberaceae family plant called fingerroot, and a rhizome part thereof is widely used for colds, enteritis, skin diseases, and urethralgia. Boesenbergia pandurata contains pinocembrin chalcone, cardamonin, pinocembrin, pinostrobin, 4-hydroxypaduratin A, and panduratin A, and these ingredients are reported to exhibit an anticancer effect (Trakoontivakorn, G., et. al., J.Arig. Food chem., 49, 3046-3050, 2001), and flavonoid-based dihydrochalcone compounds are reported to exhibit an insecticidal effect (Phytochemistry, 34, 415-419, 1993).

However, prior to the present invention, the prevention, amelioration, or treatment effect of a panduratin derivative or a Boesenbergia panurata extract in bone loss diseases including osteoporosis or alveolar bone loss has not been reported in detail.

### SUMMARY

Exemplary embodiments provide a method for ameliorating or treating a bone loss disease in a subject, the method comprising administering an effective amount of a composition comprising a Boesenbergia pandurata extract or a fraction thereof as an active ingredient to a subject in need thereof.

An exemplary embodiment provides the above mentioned method wherein the extract is an extract by at least one solvent selected from the group consisting of water, a C1-C6 organic solvent, subcritical water, and a supercritical fluid.

Another exemplary embodiment provides the above-mentioned method wherein the C1-C6 organic solvent is at least one selected from the group consisting of a C1-C6 alcohol, acetone, ether, benzene, chloroform, ethyl acetate, methylene chloride, hexane, cyclohexane, and petroleum ether.

Another exemplary embodiment provides the above-mentioned method wherein the fraction is a fraction of the Boesenbergia pandurata extract by a solvent containing at least one selected from the group consisting of hexane, chloroform, and ethyl acetate.

Still another exemplary embodiment provides the above-mentioned method wherein the extract or fraction thereof comprises at least one panduratin derivative selected from the group consisting of compounds of Chemical Formulas 1 to 3:

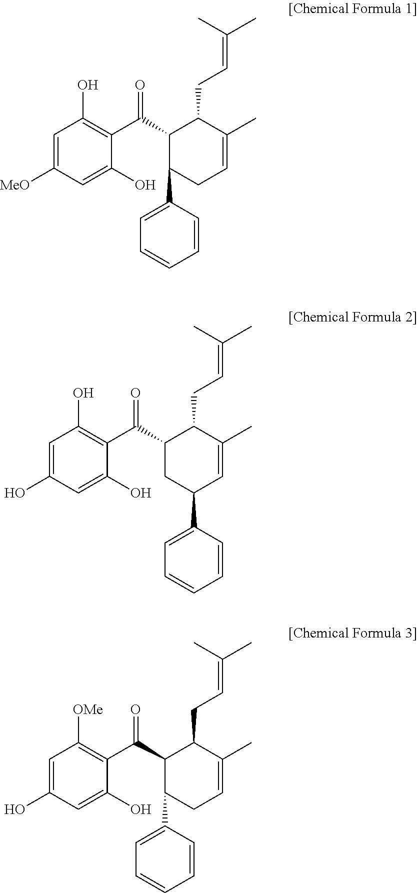 [SMILES: COc1cc(O)c(C(=O)[C@@H]2[C@H](CC=C(C)C)C(C)=CC[C@H]2c2ccccc2)c(O)c1 ; CC(C)=CC[C@@H]1C(C)=C[C@@H](c2ccccc2)C[C@@H]1C(=O)c1c(O)cc(O)cc1O ; COc1cc(O)cc(O)c1C(=O)[C@@H]1[C@@H](c2ccccc2)CC=C(C)[C@@H]1CC=C(C)C]

Still another exemplary embodiment provides the above-mentioned method wherein the bone loss disease is selected from the group consisting of osteoporosis, Paget's disease, alveolar bone loss, osteomalacia, and osteodystrophy.

Still further exemplary embodiment provides the above-mentioned method wherein the composition is a pharmaceutical composition, quasi-drug composition or food composition.

Another exemplary embodiment provides a method for ameliorating or treating a bone loss disease in a subject, the method comprising administering an effective amount of a composition comprising a panduratin derivative selected from the group consisting of compounds of Chemical Formulas 1 to 3 or a salt thereof as an active ingredient to a subject in need thereof:

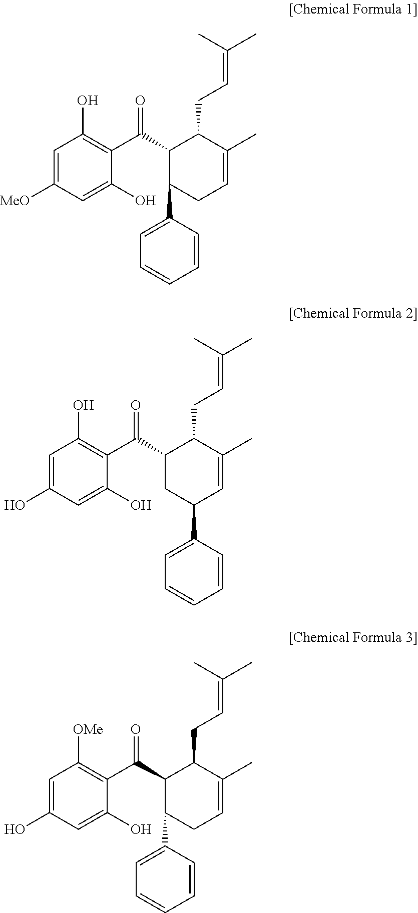 [SMILES: COc1cc(O)c(C(=O)[C@@H]2[C@H](CC=C(C)C)C(C)=CC[C@H]2c2ccccc2)c(O)c1 ; CC(C)=CC[C@@H]1C(C)=C[C@@H](c2ccccc2)C[C@@H]1C(=O)c1c(O)cc(O)cc1O ; COc1cc(O)cc(O)c1C(=O)[C@@H]1[C@@H](c2ccccc2)CC=C(C)[C@@H]1CC=C(C)C]

Still further exemplary embodiments provide the above-mentioned method wherein the panduratin derivative is extracted from Boesenbergia pandurata.

Still another exemplary embodiment provides the above mentioned method wherein the bone loss disease is selected from the group consisting of osteoporosis, Paget's disease, alveolar bone loss, osteomalacia, and osteodystrophy.

The panduratin derivative or the Boesenbergia pandurata extract of the present invention, as a natural substance, can be safely used without side effects while having an effect of treatment, prevention, or amelioration of a bone loss disease by inhibiting osteoclast differentiation, and thus can be effectively used in the preparation of a drug, a quasi-drug, a food, and the like.

### BRIEF DESCRIPTION OF THE DRAWINGS

FIG. 1 shows the results of measurement of mRNA expression of NFATc1, TRAP, and cathepsin K by the treatment with a Boesenbergia pandurata extract (BPE) in RANKL-induced RAW264.7 cells.

FIG. 2 shows the results of measurement of mRNA expression of NFATc1, TRAP, and cathepsin K by the treatment with panduratin A (Pan) in RANKL-induced RAW264.7 cells.

FIG. 3 shows the results of measurement of protein expression of NFATc1, TRAP, and cathepsin K by the treatment with a Boesenbergia pandurata extract (BPE) in RANKL-induced RAW264.7 cells.

FIG. 4 shows the results of measurement of protein expression of NFATc1, TRAP, and cathepsin K by the treatment with panduratin A (Pan) in RANKL-induced RAW264.7 cells.

FIG. 5 is a graph of measurement regarding the effect of a Boesenbergia pandurata extract (BPE) on TRAP activity in RANKL-induced RAW264.7 cells.

FIG. 6 is a graph of measurement regarding the effect of panduratin A (Pan) on TRAP activity in RANKL-induced RAW264.7 cells.

FIG. 7 shows, through microscopic analysis, the effect of a Boesenbergia pandurata extract (BPE) on osteoclast differentiation in RANKL-induced RAW264.7 cells.

FIG. 8 shows, through microscopic analysis, the effect of panduratin A (Pan) on osteoclast differentiation in RANKL-induced RAW264.7 cells.

### DETAILED DESCRIPTION

The present inventors explored and researched natural materials, which can be safely applied while having effects of restoring and maintaining the balance of bone formation and bone resorption in the bone remodeling process by inhibiting osteoclast differentiation, and as a result, the present inventors confirmed that a panduratin derivative and/or a Boesenbergia pandurata extract or a fraction thereof had effects of preventing, ameliorating, or treating a bone loss disease, and thus completed the present invention.

Hereinafter, the present invention will be described in detail.

The present invention is directed to a composition for preventing, treating, or ameliorating a bone loss disease, the composition comprising a Boesenbergia pandurata extract or a fraction thereof as an active ingredient.

Furthermore, the present invention is directed to a composition for preventing, treating, or ameliorating a bone loss disease, the composition comprising a panduratin derivative or a salt thereof as an active ingredient.

The term “Boesenbergia pandurata (Boesenbergia rotunda as the same name)” is also called fingerroot, and refers to a Zingiberaceae family plant that grows naturally in South Asia. A rhizome part thereof is widely used for colds, enteritis, skin diseases, and the like.

In the preparation of the extract of the present invention, all the parts of the fingerroot can be used, and the rhizome of Boesenbergia pandurata can be preferably used although the parts of extraction thereof are not limited. The form of the plant is not limited for the preparation of an extract, and the plant means including all of ones that have undergone processing steps, such as drying and pulverization.

In an example of the present invention, the osteoclast inhibitory effect of an extract of Boesenbergia pandurata was confirmed, and therefore, it was confirmed that the use of the extract could treat or ameliorate a bone loss disease by suppressing an excessive loss in the remodeling process of bone loss and formation to thereby balance bone loss and formation.

The Boesenbergia pandurata extract of the present invention may preferably contain a panduratin derivative. In an example of the present invention, a panduratin derivative was isolated and identified from a Boesenbergia pandurata extract, and it was experimentally confirmed that the extract as well as the panduratin compound isolated therefrom had an osteoclast differentiation inhibitory effect.

The “panduratin” is a component extracted from roots of a plant called fingerroot (Boesenbergia rotunda), and is known to have AMPK activity effect.

The panduratin derivative includes panduratin A, 4-hydroxypaduratin A, and isopanduratin A. Panduratin A is (2,6-dihydroxy-4-methoxyphenyl)[3-methyl-2-(3-methylbut-2-enyl)-5-phenylcyclohex-3-enyl]methanone and has a molecular formula of C₂₆H₃₀O₄, and may be preferably a compound having a structure of chemical formula 1 below. In addition, 4-hydroxypaduratin A is (2,4,6-dihydroxypheny[3-methyl-2-(3-methylbut-2-enyl)-6-phenylcyclohex-3-enyl]methanone and has a molecular formula of C₂₅H₂₈O₄, and may be preferably a compound having a structure of chemical formula 2 below. In addition, isopanduratin A is (2-methoxy-4,6-dihydroxyphenyl)-[3-methyl-2-(3-methylbut-2-enyl)-6-phenylcyclohex-3-enyl]methanone and has a molecular formula of C₂₆H₃₀O₄, and may be preferably a compound having a structure of chemical formula 3 below.

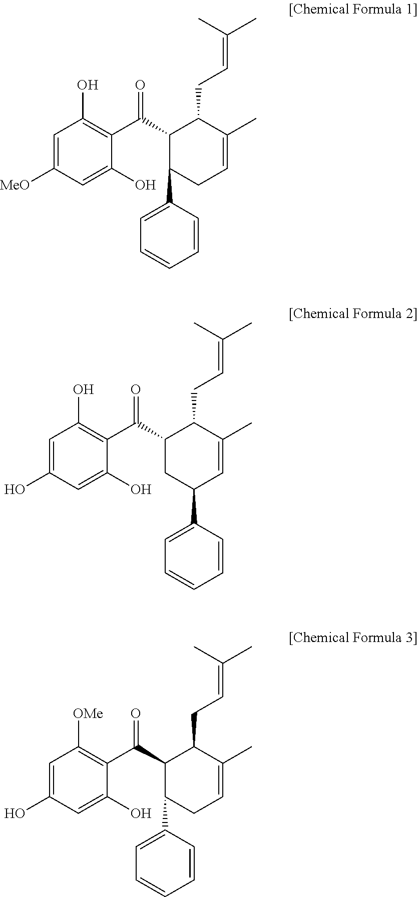 [SMILES: COc1cc(O)c(C(=O)[C@@H]2[C@H](CC=C(C)C)C(C)=CC[C@H]2c2ccccc2)c(O)c1 ; CC(C)=CC[C@@H]1C(C)=C[C@@H](c2ccccc2)C[C@@H]1C(=O)c1c(O)cc(O)cc1O ; COc1cc(O)cc(O)c1C(=O)[C@@H]1[C@@H](c2ccccc2)CC=C(C)[C@@H]1CC=C(C)C]

The composition may contain a salt of the panduratin derivative. The salt may be a pharmaceutically acceptable salt.

In an example of the present invention, an active ingredient was isolated and identified from a Boesenbergia rotunda extract, and as a result, the active ingredient was confirmed as panduratin A, and it was confirmed that the panduratin A had an osteoclast differentiation inhibitory effect. Therefore, it can be seen that the panduratin derivative of the present invention may be extracted from Boesenbergia pandurata.

As used herein, the term “extract” refers to one that is obtained by dissolving a predetermined substance in a solvent to isolate and define an active ingredient or characteristic ingredient thereof. Specifically, the term encompasses an extract extracted by adding an extraction solvent to a plant and a fraction fractionated by adding a fraction solvent to an extract prepared by extraction with an extraction solvent. Therefore, the Boesenbergia pandurata extract of the present invention means including all of an extract extracted by adding a solvent to Boesenbergia pandurata and a fraction fractionated by again adding a fraction solvent to the extract.

Specifically, the extract may be an ethanol extract, a hot-water extract, a hexane extract, an ethyl acetate extract, or a super-high pressure extract, using rhizomes of Boesenbergia pandurata.

The extraction solvent may be at least one selected from the group consisting of water, an organic solvent, subcritical water, and a supercritical fluid. The organic solvent may be a polar solvent, a non-polar solvent, a polar and non-polar mixture solvent, or water. Specifically, the solvent may be any one selected from the group consisting of a C1-C6 alcohol, acetone, ether, benzene, chloroform, ethyl acetate, methylene chloride, hexane, cyclohexane, petroleum ether, and water.

In an example of the present invention, an extract was prepared by adding ethanol to Boesenbergia pandurata, and the ethanol extract of Boesenbergia pandurata was confirmed to have an effect of inhibiting osteoclast activity. Therefore, ethanol can be preferably used as an extraction solvent.

The plant extract may be prepared by a conventional method for preparing a plant extract. More specifically, a method wherein an extraction solvent is added to the plant with impurities removed and an extraction process is executed may be carried out. The extraction process may be cold extraction, warm extraction, pressurized extraction, or ultrasonic pulverization extraction. For example, the extract can be obtained by extraction and purification of dried plant bodies with purified water, ethanol, subcritical water, or supercritical carbon dioxide, which is suitable for food processing, by extraction and purification of Boesenbergia pandurata plant under super-high pressure conditions of 100 MPa or higher using a super-high pressure extraction apparatus, or by isolation and purification of the oil obtained from direct compression of plant bodies.

The fraction solvent may be water, butanol, ethyl acetate, chloroform, hexane, or a mixture thereof. The fraction may be an extract prepared by the above extraction method, specifically, a fraction obtained by further performing a fractionation process a crude extract. The fraction solvent may be a solvent selected from the group consisting of ethyl acetate, ether, chloroform, benzene, hexane, methylene chloride, and mixed solvents thereof. Specifically, the fractionation process may be performed by sequentially adding mixed solvents to the crude extract and then sequentially obtaining the layer-separated fractions.

In one embodiment of the present invention, the extract may be a first fraction by hexane-chloroform-ethyl acetate, fractionated by adding mixed solvents of hexane, chloroform, and ethyl acetate to an ethanol extract of Boesenbergia pandurata. In addition, the extract may be a second fraction of hexane-ethyl acetate-methanol, fractionated from the first fraction by again using, as development solvents, hexane, ethyl acetate, and methanol. The mixed solvents of hexane, chloroform, and ethyl acetate may be obtained by mixing hexane, chloroform, and ethyl acetate at a volume ratio of (1-5):1:(0.1-0.5), and may be obtained by mixing hexane, ethyl acetate, and methanol at a volume ratio of (15-20):(0.5-4):1. The fraction of the present invention contains a largest amount of panduratin component, and thus has excellent effects of preventing, treating, or ameliorating a bone loss disease.

The extract or fraction may be concentrated or the solvents were removed therefrom by, after the extraction or fractionation, conducting vacuum filtering or further conducting concentration and/or freeze-drying. The obtained extract can be stored in a deep freezer until used.

The fractionation is conducted using techniques such as solvent fractionation, silica gel chromatography, prep-HPLC, and the like, thereby preparing a particular fraction having concentrated active substances.

As used herein, the term “bone loss” refers to a symptom of bone loss caused by imbalance of osteoclasts and osteoblasts, and the term “bone loss disease” means including all diseases related to the symptom. Therefore, the term encompasses all the diseases caused by a low bone density resulting from the loss of bone due to excessive activity of osteoclasts or caused by non-smooth bone generation due to decreased activity of osteoblasts. Specific examples of the disease include osteoporosis, Paget's disease, alveolar bone loss, osteomalacia, and osteodystrophy.

Osteoporosis refers to a condition in which the strength of bone is weakened due to a reduced amount of bone and a qualitative change thereof, and thus fracture is highly likely to occur, and the symptoms can be alleviated, ameliorated, or treated or corresponding diseases can be prevented by lowering functions of osteoclasts or activating osteoblasts.

Paget's disease refers to a bone disease in which bone remodeling is excessively advanced to invade a wide area of skeletal systems, and the symptoms can be alleviated, ameliorated, or treated or corresponding diseases can be prevented by inhibiting bone resorption and/or bone formation.

The term means including all the symptoms in which the bone in the alveolar bone loss periodontium is lost. The Paget's disease may be caused by periodontitis or gingivitis, but is not limited thereto.

Osteomalacia refers to a disease in which a reduction of bone density is shown due to abnormal calcification of newly formed bone matrix, which is also called rickets. The inhibition of osteoclast activity can delay ossifluence or prevent the fracture by ossifluence.

Osteodystrophy is the inhibition of bone growth caused by nutritional imbalance, and may be accompanied by osteoporosis. Osteodystrophy is also called a bone formation disorder or an osteoarthritis disorder. Osteodystrophy includes renal osteodystrophy, hypertrophic osteodystrophy, and chondrodystrophia fetalis (fetal chonchodystrophy).

In an example of the present invention, it was experimentally confirmed that the treatment of osteoclasts with panduratin or the extract of the present invention inhibited osteoclast activity. Therefore, the composition of the present invention is effective in the preventing, treating, or ameliorating a bone loss disease by inhibiting osteoclast activity to inhibit and delay excessive bone resorption causing a bone loss.

As used herein, the term “prevention” or “preventing” refers to all the acts that inhibit or delay the occurrence of diseases or symptoms. In the present invention, the term refers to delaying the period of occurrence of a bone loss disease or inhibiting the occurrence of a disease by inhibiting the activity of osteoclasts.

As used herein, the term “amelioration” or “ameliorating” refers to all the acts that favorably or advantageously change a disease or symptom condition. In the present invention, the term refers to the amelioration of a symptom of osteoporosis or a symptom such as alveolar bone loss through an action of inhibiting osteoclast activity.

As used herein, the term “treatment” or “treating” refers to inhibition of disease development, inhibition of recurrence, alleviation of symptoms, reduction of direct or indirect pathological consequences of disease, a reduction in the rate of disease progression, an improvement in the disease state, an improvement, or alleviation.

In an example of the present invention, it was confirmed that the treatment with a Boesenbergia pandurata extract or panduratin could inhibit osteoclast differentiation causing osteoporosis.

Therefore, in such an aspect, the composition of the present invention can be a pharmaceutical composition for preventing or treating a bone loss disease.

The composition of the present invention may further contain, in addition to the foregoing effective ingredient, at least one active ingredient having the same or similar functions, for the prevention or treatment of a bone loss disease. For example, the composition may further contain a known therapeutic agent for osteoporosis, a known therapeutic agent for an alveolar bone disease, and the like, which are effective in the prevention or treatment of a bone loss disease. The composition of the present invention, when containing an additional ingredient, can have further enhanced effects of preventing or treating a bone loss disease. The addition of the addition ingredient may require a consideration of stability due to the complex use, ease of formulation, and stability of active ingredients. The additional ingredient may be contained in a content of 0.0001 wt % to 10 wt % inclusive, relative to the weight of the entire composition. For example, the additional ingredient may be contained in a content of 0.0001 wt % to 1 wt % inclusive, 0.0001 wt % to 0.1 wt % inclusive, 0.0001 wt % to 0.001 wt % inclusive, 0.001 wt % to 10 wt % inclusive, 0.001 wt % to 1 wt % inclusive, 0.001 wt % to 0.1 wt % inclusive, 0.01 wt % to 10 wt % inclusive, and 0.01 wt % to 1 wt % inclusive. The above content range may be adjusted according to conditions, such as safety, and ease of formulation of the Boesenbergia pandurata extract or panduratin compound of the present invention.

The pharmaceutical composition of the present invention may further a pharmaceutically acceptable salt of panduratin. As used herein, the term “pharmaceutically acceptable” refers to being physiologically acceptable, and not usually causing an allergic response or a similar response when administered to humans. An acid addition salt formed by a pharmaceutically acceptable free acid is preferable as the salt.

The pharmaceutically acceptable salt may be an acid addition salt formed by using an organic acid or inorganic acid. Examples of the organic acid include formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranyl acid, dichloroacetic acid, aminooxy acetic acid, benzensulfonic acid, p-toluenesulfonic acid, or methanesulfonic acid. Examples of the inorganic acid include hydrochloric acid, bromic acid, sulfuric acid, phosphoric acid, nitric acid, carbonic acid, or boric acid. The acid addition salt may be preferably in the form of a chloric acid salt or acetic acid salt, and more preferably in the form of a chloric acid salt.

The above-mentioned acid addition salt may be prepared by a general salt preparation method, including: a) directly mixing panduratin and an acid; b) dissolving one of the same in a solvent or a hydrated solvent, followed by mixing; or c) adding panduratin to an acid in a solvent or a hydrated solvent, followed by mixing.

Separately as above, additional examples of the acceptable salt include a GABA salt, a gabapentin salt, a pregabalin salt, a nicotine salt, an adipate salt, a hemi-malonic acid salt, a cysteine salt, an acetyl cysteine salt, a methionine salt, an arginine salt, a lysine slat, an ornithine salt, or an aspartic acid salt.

In addition, the pharmaceutical composition of the present invention may further contain a pharmaceutically acceptable salt.

Examples of the pharmaceutically acceptable carrier may further include a carrier for oral administration or a carrier for parenteral administration. The carrier for oral administration may include lactose, starch, a cellulose derivative, magnesium stearate, stearic acid, and the like. The carrier for parenteral administration may include water, proper oil, a saline solution, aqueous glucose, glycol, and the like. In addition, the carrier may further include a stabilizer and a preservative. Suitable examples of the stabilizer include an antioxidant, such as sodium hydrogen sulfite, sodium sulfite, or ascorbic acid. Suitable examples of the preservative include benzalkonium chloride, methyl- or propyl-paraben, and chlorobutanol. Other pharmaceutically acceptable carriers are referenced in the following literature (Remington's Pharmaceutical Sciences, 19th ed., Mack Publishing Company, Easton, Pa., 1995).

The composition of the present invention may be administered to mammals including humans by any method. The pharmaceutical composition of the present invention may be administered orally or parentally, and examples of parental administration may be, but is not limited to, intravenous, intramuscular, intraarterial, intramedullary, intradural, intracardiac, transdermal, subcutaneous, intraperitoneal, intranasal, intestinal, topical, sublingual, or rectal administration.

The pharmaceutical composition of the present invention may be formulated as a preparation for oral administration or parental administration according to the route of administration as described above. The composition according to the present invention may be formulated by using at least one buffer (for example, a saline solution or PBS), a carbohydrate (for example, glucose, mannose, sucrose, or dextran), an antioxidant, a bacteriostat, a chelating agent (for example, EDTA or glutathione), a filler, an extender, a binder, an adjuvant (for example, aluminum hydroxide), a suspending agent, a thickener, a wetting agent, a disintegrant, or a surfactant, a diluting agent, or an excipient.

A solid preparation for oral administration includes a tablet, a pill, a powder, granules, a liquid, a gel, syrup, slurry, a suspension, or a capsule, and such a solid preparation may be prepared by mixing le, a troche, and the like. These solid preparations may be prepared by mixing, with the pharmaceutical composition of the present invention, at least one excipient, such as starch (including corn starch, wheat starch, rice starch, and potato starch), calcium carbonate, sucrose, lactose, dextrose, sorbitol, mannitol, xylitol, erythritol maltitol, cellulose, methylcellulose, sodium carboxymethyl cellulose, hydroxypropylmethyl-cellulose, or gelatin. For example, a tablet or a sugar-coated tablet may be obtained by mixing an active ingredient with a solid excipient, pulverizing the mixture, adding a suitable adjuvant thereto, and then processing the mixture into a granule mixture.

In addition to simple excipient, lubricants such as magnesium stearate and talc may be used. A liquid preparation for oral administration corresponds to a suspending agent, a liquid for internal use, an emulsion, a syrup, and the like, and may include, in addition to water or liquid paraffin as a simple diluent, several excipients, for example, a wetting agent, a sweetening agent, an aroma, and a preservative.

In some cases, cross-linked polyvinyl pyrrolidone, agar, alginic acid, or sodium alginate may be added as a disintegrant, and an anti-coagulant, a lubricant, a wetting agent, a flavoring agent, an emulsifier, and a preservative may be further added.

As for the parenteral administration, the pharmaceutical composition of the present invention may be formulated in a dosage form of an injection, a transdermal administration preparation, and a nasal inhalant, together with a suitable parenteral carrier, by a method known in the art. The injection needs to be essentially sterilized, and needs to be protected from the contamination of microorganisms, such as bacteria and fungus. Examples of the suitable carrier for an injection may include, but are not limited to, water, ethanol, a polyol (e.g., glycerol, propylene glycol, liquid polyethylene glycol, etc.), a mixture thereof, and/or a solvent or dispersing medium including vegetable oil. More preferably, Hanks' solution, Ringer's solution, phosphate buffered saline (PBS) or sterile water containing triethanolamine for injection, or an isotonic solution (such as 10% ethanol, 40% propylene glycol, or 5% dextrose) may be used as a suitable carrier. In order to protect the injection from microbial contamination, the injection may further containing various antibacterial and antifungal agents, such as paraben, chlorobutanol, phenol, sorbic acid, and thimerosal. In most cases, the injection may further contain an isotonic agent, such as sugar or sodium chloride.

The form of the transdermal administration preparation includes ointment, cream, lotion, gel, solution for external application, plaster, liniment, and aerosol. The “transdermal administration” means locally administering a pharmaceutical composition to skin to deliver an effective amount of an active ingredient through the skin.

In the case of an inhalation agent, the compound used according to the invention may be conveniently delivered in the form of aerosol spray from a pressurized pack or a nebulizer, using a suitable propellant, for example, dichlorofluoromethane, trichlorofluoromethane, dichlorotetrafluoroethane, carbon dioxide, or other suitable gases. In the case of a pressurized aerosol, the unit of administration may be determined by providing a valve that delivers a measured quantity. For example, a gelatin capsule and a cartridge used in an inhaler or an insufflator may be formulated to contain a compound, and a powder mixture of proper powder materials, such as lactose or starch. These preparations are described in the literature, which is a formulary generally known in pharmaceutical chemistry (Remington's Pharmaceutical Science, 15th Edition, 1975. Mack Publishing Company, Easton, Pa. 18042, Chapter 87: Blaug, Seymour).

The pharmaceutical composition of the present invention, when containing panduratin or a Boesenbergia pandurata extract or a fraction thereof in an effective amount, can provide preferable effects of preventing, ameliorating, or treating a bone loss disease. As used herein, the term “effective amount” refers to an amount that exhibits a greater response than a negative control, and preferably an amount sufficient to prevent, ameliorate, or treat a bone loss disease. The pharmaceutical composition of the present invention may contain 0.01-99.99% of panduratin or a Boesenbergia pandurata extract and the balance pharmaceutical acceptable carrier. The effective amount of panduratin or a Boesenbergia pandurata extract or fragment thereof contained in the pharmaceutical composition of the present invention may vary according to the form in which the composition is productized.

A total effective amount of the pharmaceutical composition of the present invention may be administered to a patient in a single dose, or may be administered in a multiple dose for a long period of time by a fractionated treatment protocol. In the pharmaceutical composition of the present invention, the content of the active ingredient may vary depending on the severity of a disease. For example, the pharmaceutical composition of the present invention may be administered once or divided into multiple doses such that panduratin or a Boesenbergia pandurata extract is administered in an amount of preferably 0.001-100 mg, and more preferably 0.01-10 mg per 1 kg of body weight a day. However, as for the dosage of panduratin or a Boesenbergia pandurata extract, an effective dose thereof with respect to a patient is determined in consideration of various factors, such as route of administration, number of times of treatment, patient's age, body weight, health condition, and sex, severity of disease, food, and excretion rate, and therefore, considering this fact, a person skilled in the art could determine a proper effective amount of the panduratin or the Boesenbergia pandurata extract according to a particular use for preventing, treating, or ameliorating bone loss. The pharmaceutical composition according to the present invention is not particularly limited to the dosage form, route of administration, and administration method thereof as long as the composition shows the effects of the present invention.

The pharmaceutical composition for preventing or treating a bone loss disease of the present invention may be used alone or in combination with other methods employing surgery, radiation therapy, hormone therapy, chemical therapy, or a biological response controller.

The pharmaceutical composition for preventing or treating a bone loss disease of the present invention may be provided in a dosage form of an externally applied preparation containing, as an active ingredient, panduratin or a Boesenbergia pandurata extract. In this aspect, the composition of the present invention may be a quasi-drug composition for preventing or ameliorating bone loss and a quasi-drug containing the same.

The externally applied preparation may be directly applied onto the skin or into the oral cavity. The pharmaceutical composition for preventing or treating a bone loss disease of the present invention, when used as an externally applied preparation, may further contain an adjuvant that is commonly used in the field of dermatology, for example, a fatty substance, an organic solvent, a solubilizing agent, a concentrating agent, a gelling agent, a softening agent, an antioxidant, a suspending agent, a stabilizer, a forming agent, an aroma, a surfactant, water, an ionic or nonionic emulsifier, a filler, a sequestering agent, a chelating agent, a preservative, vitamins, a blocker, a wetting agent, essential oil, a dye, a pigment, a hydrophilic or lipophilic activator, a lipid vesicle, or any other ingredient conventionally used in an externally-applied dermal preparation. In addition, the above ingredients may be introduced in amounts that are generally used in cosmetology.

The preparation of the present invention, when provided as an externally-applied preparation, may be in a dosage form of a liquid preparation, ointment, a patch, gel, cream, or aerosol, but is not limited thereto. According to an embodiment of the present invention, the quasi-drug of the present invention may include oral care products including toothpaste, gargle, and mouse spray, ointment, masks, poultices, patches, and percutaneous absorbers.

In an example of the present invention, the treatment with panduratin or a Boesenbergia pandurata extract inhibited osteoclast differentiation, and thus when the active ingredients are applied to oral care products, the oral care products show an effect of preventing or ameliorating an alveolar bone disease caused by excessive bone resorption. Therefore, the quasi-drug composition may be an oral care composition for preventing or ameliorating bone loss.

The composition of the present invention, when used as a quasi-drug composition, can be properly used according to the conventional method by adding panduratin or a Boesenbergia pandurata extract as it is or together with other quasi-drug ingredients. The amounts of active ingredients mixed may be properly determined according to the purpose of use (prevention, health, or therapeutic treatment).

The contents of the pharmaceutical composition above and the food composition below may apply to the quasi-drug composition and quasi-drug of the present invention.

In another aspect, the composition of the present invention may be a food composition for preventing or ameliorating bone loss.

The food composition may be a health functional food composition.

In an example of the present invention, it was confirmed that the treatment with panduratin or a Boesenbergia pandurata extract inhibited osteoclast differentiation and activity, and thus could directly prevent or delay bone loss. The bone loss may be caused by a disease causing bone loss, preferably by a bone loss disease resulting from excessive activity of osteoclasts.

The food composition of the present invention encompasses all food types including a functional food, a nutritional supplement, a health food, a food additive, and an animal feed, and the food composition are taken by humans or animals including livestock.

The above types of food composition may be prepared in various forms according to the conventional methods known in the art. The food composition may be prepared by adding the panduratin or the Boesenbergia pandurata extract to general food including, but is not limited to, beverages (including alcoholic beverages), fruits and processed foods thereof (e.g., canned fruit, bottled food, jam, marmalade, etc.), fishes, meats and processed products thereof (e.g., ham, sausage, corned beef, etc.), breads, noodles (e.g., udong, buckwheat noodles, ramen, spaghetti, macaroni, etc.), fruit juices, a variety of drinks, cookies, syrups, dairy products (e.g., butter, cheese, etc.), edible vegetable oils, margarine, vegetable proteins, retort foods, frozen foods, and various seasonings (e.g., soybean paste, soybean sauce, sauces, etc.).

Alternatively, a nutritional supplement may be prepared by adding the panduratin or the Boesenbergia pandurata extract to a capsule, tablet, pill, or the like, but is not limited thereto.

As for health functional food, the panduratin or the Boesenbergia pandurata extract may be, for example, taken by liquefaction, granulation, encapsulation, and powdering such that the panduratin or the Boesenbergia pandurata extract itself can be drunk (as a health drink) in the form of tea, juice, and drink, but is not limited thereto. In addition, the panduratin or the Boesenbergia pandurata extract may be prepared in the form of a powder or a concentrate for the use as a form of a food additive. In addition, the panduratin or the Boesenbergia pandurata extract may be prepared in the form of a composition by mixing with an active ingredient that is known to have an effect of preventing or ameliorating bone loss.

When the food composition of the present invention is used as a health drink food composition, the corresponding health drink may contain, as additive ingredients, several flavoring agents or natural carbohydrates like in ordinary drinks. The foregoing carbohydrates may be: monosaccharides, such as glucose and fructose; disaccharides such as maltose and sucrose; polysaccharides such as dextrin and cyclodextrin; and sugar alcohols, such as xylitol, sorbitol, and erythritol. The sweetening agent may include: natural sweetening agents, such as thaumatin and stevia extracts; and synthetic sweetening agents, such as saccharin and aspartame. The proportion of the natural carbohydrate is generally about 0.01-0.04 g, and preferably about 0.02-0.03 g per 100 mL of the composition of the present invention.

Panduratin or a Boesenbergia pandurata extract may be contained as an active ingredient of a food composition for preventing or ameliorating bone loss, and the amount thereof is an amount sufficient to obtain an effect of preventing or ameliorating a bone loss disease, and is not particularly limited. However, the content thereof is preferably 0.01-100 wt % relative to a total weight of the entire composition. The food composition of the present invention can be prepared by mixing panduratin or a Boesenbergia pandurata extract with another active ingredient that is known to have an effect of preventing or ameliorating bone loss.

Furthermore, the health food of the present invention may contain various nutrients, vitamins, electrolytes, flavoring agents, colorants, pectic acid and salts thereof, alginic acid and salts thereof, organic acids, protective colloid thickeners, pH adjusters, stabilizers, stabilizing agents, preservatives, glycerin, alcohols, or carbonating agents. In addition, the health food of the present invention may contain fruit flesh for manufacturing natural fruit juice, fruit juice drink, and vegetable drink. These ingredients may be used independently or in mixture. The proportion of such an additive is not greatly important, but is generally selected within a range of 0.01-0.1 parts by weight relative to 100 parts by weight of the composition of the present invention.

Some embodiments according to the present invention provide a method for ameliorating or treating a bone loss disease in a subject, the method comprising administering an effective amount of a composition comprising a Boesenbergia pandurata extract or a fraction thereof as an active ingredient to a subject in need thereof.

Another embodiments according to the present invention provide a method for ameliorating or treating a bone loss disease in a subject, the method comprising administering an effective amount of a composition comprising a panduratin derivative selected from the group consisting of compounds of Chemical Formulas 1 to 3 or a salt thereof as an active ingredient to a subject in need thereof:

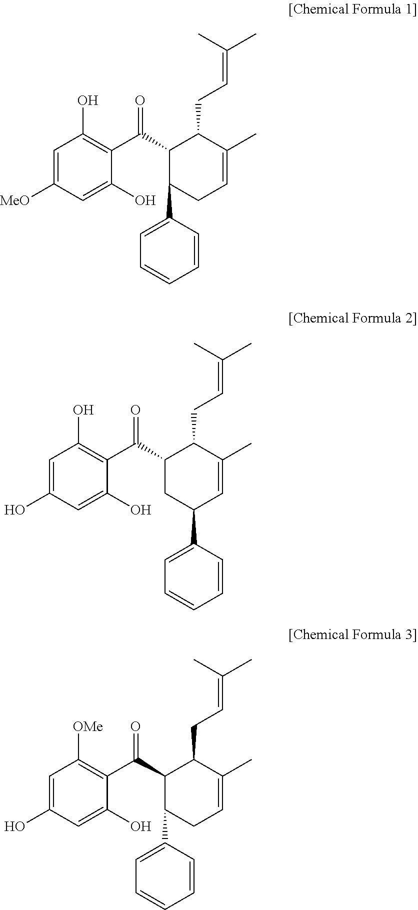 [SMILES: COc1cc(O)c(C(=O)[C@@H]2[C@H](CC=C(C)C)C(C)=CC[C@H]2c2ccccc2)c(O)c1 ; CC(C)=CC[C@@H]1C(C)=C[C@@H](c2ccccc2)C[C@@H]1C(=O)c1c(O)cc(O)cc1O ; COc1cc(O)cc(O)c1C(=O)[C@@H]1[C@@H](c2ccccc2)CC=C(C)[C@@H]1CC=C(C)C]

The term “effective amount” of the present invention refers to an amount that, when administered to a subject, leads to the improvement, alleviation, treatment, or prevention of a bone loss disease. The term “subject” may be an animal, preferably a mammal including humans and livestock, animal-derived cells, tissues, or organs. The subject may be a patient or livestock needed for treatment.

Hereinafter, the present invention will be described in detail with reference to examples. The present invention can be variously modified and can have several forms, and thus, it is to be understood that the particular examples and explanation described below are for illustrative purposes only and the present invention is not limited to particular embodiments. It is to be understood that the scope of the present invention is intended to cover all the modifications, equivalents or substitutions, included in the technical idea and technical scope of the present invention.

### Example 1: Preparation of Boesenbergia Pandurata Extract

After dried Boesenbergia pandurata was crushed in a mixer, 100 g of the crushed Boesenbergia pandurata sample was extracted in 500 mL of ethanol with stirring at 50° C. for 30 minutes. The extracted sample was filtered twice through Whatman No. 2 filter paper, and the filtered extract was concentrated in a vacuum rotary evaporator to remove solvent components, followed by freeze-drying to remove water, thereby obtaining a Boesenbergia pandurata extract.

### Example 2: Isolation and Determination of Structure of Panduratin A

### Example 2-1: Isolation of Panduratin A

The concentrated Boesenbergia pandurata extract obtained in Example 1 was mixed with ethyl acetate to extract an ethyl acetate soluble component, and ethyl acetate was removed under reduced pressure to concentrate only the ethyl acetate soluble component. Then, the concentrated component was loaded into a column packed with 6×15 cm silica gel, and was fractionated using a solvent system containing hexane, chloroform, and ethyl acetate mixed with each other at a ratio of 15:5:1.5 (v/v/v). The concentrated component was divided into a total of six fractions according to the order of fractionation, and each of the fractions was concentrated and dried. Among the six fractions, No. fraction (fraction 3) was subjected to thin film chromatography (TLC, silica gel 60F254, Merck) using a developing solvent containing hexane, ethyl acetate and methanol mixed with each other at a ratio of 18:2:1 (v/v/v) to obtain a total of three fractions according to the order of fractionation, and the fractions were concentrated and dried. Among the three fractions, No. 2 fraction (fraction 3-2) was subjected to recycling high-performance liquid chromatography (recycling HPLC, column: W252, 20.0 mm ID×500 mm L) to obtain a total of two fractions according to the order of fractionation, and each of the fractions was concentrated and dried. Finally, among the two fractions, No. 2 fraction (fraction 3-2-2) was concentrated and dried, thereby isolating a single pure active substance.

### Example 2-2: Determination of Structure of Panduratin A

In order to determine the structure of the single active substance isolated in Example 2-1, the ¹H-NMR spectrum and ¹³C-NMR spectrum of the single active substance were measured at 500 MHz and 125 MHz (solvent: CDCl₃), respectively. In order to measure correlation and ¹H-¹³C correlation on the basis of the results of the obtained ¹³C-NMR spectrum and ¹H-NMR spectrum, the ¹H-¹H COSY spectrum and ¹H-¹³C HSQC spectrum of the active substance were measured, and the signal of each carbon was identified using a wavelength from carbon resonance.

In addition, for the mass spectrometry of the isolated single substance, the EI/MS was measured. In the EI/MS, [M⁺H⁺] was observed at m/z 407, and thus this compound was found to have a molecular weight of 406 and a molecular formula of C₂₆H₃₀O₄.

The results of ¹H-NMR, ¹³C-NMR, ¹H-¹H COSY, 1H-13C HSQC, and EI/MS were analyzed comparatively with a previous study report (Phytochemistry, 26: 1542-1543, 1987). As a result, it was confirmed that the single substance isolated in Example 2-1 was (2,6-dihydroxy-4-methoxyphenyl)[3-methyl-2-(3-methylbut-2-enyl)-5-phenylcyclohex-3-enyl]methanone which is a panduratin A compound represented by the following formula 1:

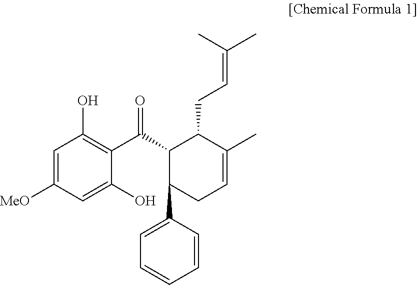 [SMILES: COc1cc(O)c(C(=O)[C@@H]2[C@H](CC=C(C)C)C(C)=CC[C@H]2c2ccccc2)c(O)c1]

### Test Example 1: Confirmation of Osteoclast Differentiation Inhibitory Effect of Boesenbergia Pandurata Extract and Panduratin A

### Test Example 1-1: Confirmation of Osteoclast Differentiation Inhibitory Effect of Boesenbergia Pandurata Extract and Panduratin A through RT-PCR

RAW264.7 osteoclast precursor cells (American Type Culture Collection (ATCC), Manassas, Va., USA) were seeded at 8×10⁴ cells/well in 6-well microtiter plates using Dulbecco's modified Eagle's medium (DMEM, Hyclone, Logan, Utah, USA) containing 10% bovine fetal serum. After 24 hours, α-modified Eagle's medium (α-MEM, Gibco, Grand Island, N.Y., USA) containing 10% bovine fetal serum was treated with 25 ng/ml RANKL (Peprotech Inc., Rocky Hill, N.J., USA) and Boesenbergia pandurata extract (1 μg/ml and 10 μg/ml) or panduratin A (0.1 μM and 1 μM) obtained in Example 2, respectively, thereby inducing differentiation for 4 days. The medium was exchanged every two days.

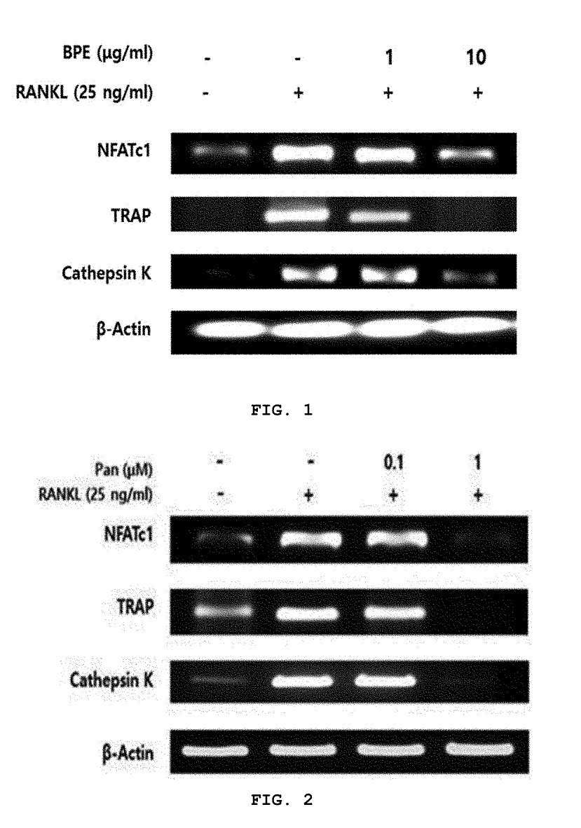

In order to examine the mRNA expression levels of nuclear factor of activated T-cells (NFATc1, cytoplasmic 1) and the enzyme TRAP, cathepsin K, which are major transcription factors expressed in osteoclast differentiation, RT-PCR was performed. Total RNA was harvested from the differentiated cells by Trizol reagent (Takara, Tokyo, Japan), and for cDNA synthesis, the RNA was subjected to reverse transcription, and then RT-PCR was performed using the following primers. The results are shown FIG. 1 or 2.

β-Actin:
Forward primer (SEQ ID NO. 1):
5′-CAGCTCAGTAACAGTCCGCC-3′
Reverse primer (SEQ ID NO. 2):
5′-TCACTATTGGCAACGAGCGG-3′
NFATc1:
Forward primer (SEQ ID NO. 3):
5′-CCTGGAGATCCCGTTGCTTC-3′
Reverse primer (SEQ ID NO. 4):
5′-TCCCGGTCAGTCTTTGCTTC-3′
TRAP:
Forward primer (SEQ ID NO. 5):
5′-AAATCACTCTTTAAGACCAG-3′
Reverse primer (SEQ ID NO. 6):
5′-TTATTGAATAGCAGTGACAG-3′
Cathepsin K:
Forward primer (SEQ ID NO. 7):
5′-ATCTCTCTGTACCCTCTGCA-3′
Reverse primer (SEQ ID NO. 8):
5′-CCTCTCTTGGTGTCCATACA-3′

As shown in FIG. 1 or 2, it could be confirmed that the mRNA expression levels of NFATc1, TRAP, and cathepsin K were reduced in the osteoclasts differentiated by the treatment with the Boesenbergia pandurata extract and panduratin A. It could be especially confirmed that the mRNA expression of TRAP and cathepsin K was hardly shown in the Boesenbergia pandurata extract (10 μg/ml) treatment group or the panduratin A (1 μM) treatment group. These results indicate that the Boesenbergia pandurata extract and panduratin A of the present invention has excellent effect of inhibiting the bone resorption of osteoclasts.

### Test Example 1-2: Confirmation of Osteoclast Differentiation Inhibitory Effect of Boesenbergia Pandurata Extract and Panduratin A through Western Blot

RAW264.7 osteoclast precursor cells (ATCC) were seeded at 1×10⁵ cells/well in 6-well microtiter plates using DMEM (Hyclone) containing 10% bovine fetal serum. After 24 hours, a-MEM (Gibco) containing 10% bovine fetal serum was treated with 25 ng/ml RANKL (Peprotech) and Boesenbergia pandurata extract (1 μg/ml and 10 μg/ml) or panduratin A (0.1 μM and 1 μM), respectively, followed by incubation for 24 hours. In order to examine the protein expression levels of NFATc1 and the enzyme TRAP, cathepsin K, which are major transcription factors expressed in osteoclast differentiation, western blotting was performed.

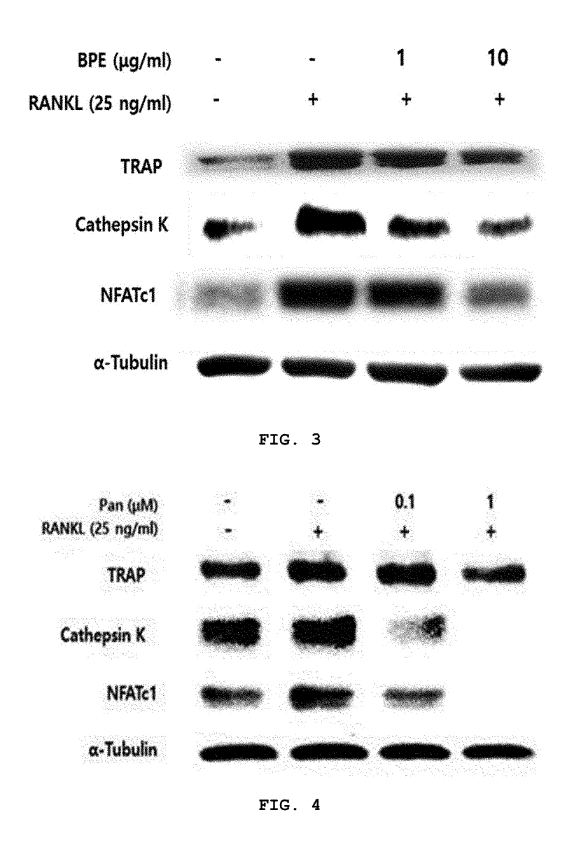

Specifically, the cells were lysed with NP40 buffer (ELPIS-Biotech, Daejeon, Korea) containing protease inhibitor cocktail. The cells lysed in the buffer were transferred into 1.5 ml-tubes, followed by centrifugation at 13,000 rpm for 10 minutes, thereby obtaining only supernatant. The supernatant was quantified using the Bradford assay (Bio-Rad Laboratories Inc., Hercules, Calif., USA). The quantified proteins were boiled for 5 minutes, and then separated by electrophoresis using 10% SDS-PAGE, and the separated proteins were transferred onto nitrocellulose membranes. Primary antibodies against NFATcl, TRAP, and cathepsin K were diluted with 2.5% bovine serum albumin (BSA) at a ratio of 1:1000, and incubated together with the proteins transferred onto the nitrocellulose membranes at room temperature for 20 hours. After the incubation with the primary antibodies, the nitrocellulose membranes were washed three times with Tris-buffer Saline Tween 20 (TBST) for 10 minutes. After the washing, secondary antibody recognizing the primary antibodies was diluted with 2.5% BSA at 1:5000, and incubated with the nitrocellulose membranes at room temperature for 2 hours, followed by washing three times with TBST for 10 minutes for each time. Protein bands were developed with ECL western blotting detection reagents (Amersham, Tokyo, Japan), and the developed protein bands were visualized with G;BOX EF imaging system (Syngene, Cambridge, UK). The results are shown in FIG. 3.

As shown in FIG. 3 or 4, it could be confirmed that the protein expression levels of NFATc1, TRAP, and cathepsin K were reduced in the osteoclasts differentiated by the treatment with the Boesenbergia pandurata extract or panduratin A. Especially, the protein expression of NFATc1 and cathepsin K was hardly shown in the Boesenbergia pandurata extract (10 μg/ml) treatment group or the panduratin A (1 μM) treatment group. These results indicate that the Boesenbergia pandurata extract or panduratin A of the present invention has excellent effect of inhibiting the bone resorption of osteoclasts.

### Test Example 1-3: Verification of TRAP Enzyme Activity Inhibitory Effect of Boesenbergia Pandurata Extract or Panduratin A

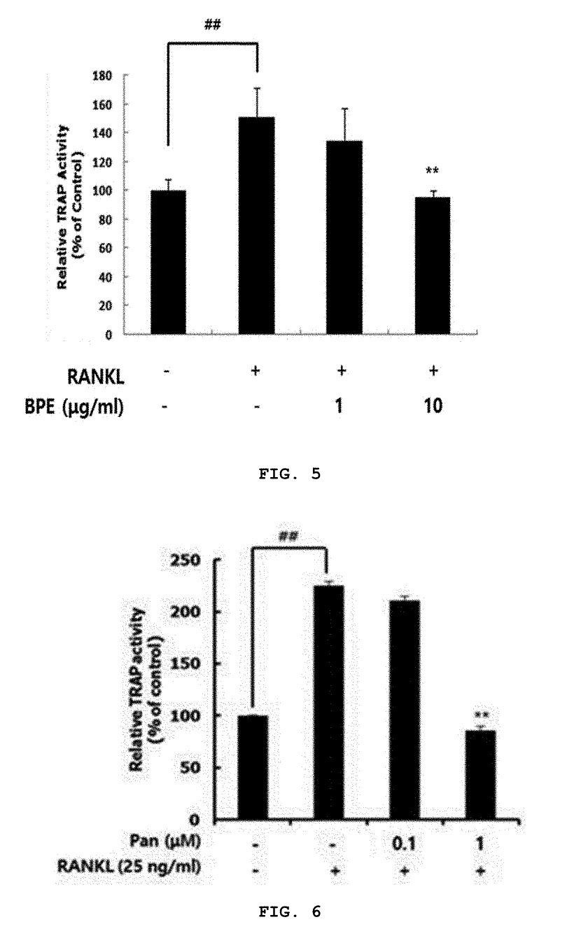

Using the same method as in Test example 1, RAW264.7 osteoclast precursor cells were attached in 6-well microtiter plates, and then α-MEM containing 10% bovine fetal serum was treated with 25 ng/ml RANKL (Peprotech) and Boesenbergia pandurata extract (1 μg/ml and 10 μg/ml) or panduratin A (0.1 μM and 1 μM) obtained in Example 2, respectively, thereby inducing differentiation for 4 days. The medium was exchanged every two days. Thereafter, supernatant was completely removed, and the cells were lysed with 0.1% Triton X-100 (Oriental chemical industries, Seoul, Korea). The cells lysed in the buffer were transferred into 1.5 ml-tubes, followed by centrifugation at 13,000 rpm for 15 minutes, thereby obtaining only supernatant. The supernatant containing TRAP was analyzed using a leukocyte acid phosphatase kit (Sigma-Aldrich, St. Louis, Mo., USA) according to manufacturer's guide line. The results are shown FIG. 5 or 6.

As shown in FIG. 5 or 6, it could be confirmed that TRAP enzyme activity was significantly reduced in the osteoclasts differentiated by the treatment with the Boesenbergia pandurata extract or panduratin A (##p<0.01, **p<0.01). It was especially confirmed that the TRAP enzyme activity was lowered in the Boesenbergia pandurata extract (10 μg/ml) treatment group or the panduratin A (1 μM) treatment group. These results indicate that the Boesenbergia pandurata extract or panduratin A of the present invention has excellent effect of inhibiting the bone resorption of osteoclasts.

### Test Example 1-4: Confirmation of Osteoclast Differentiation Inhibitory Effect of Boesenbergia Pandurata Extract and Panduratin A through TRAP Staining

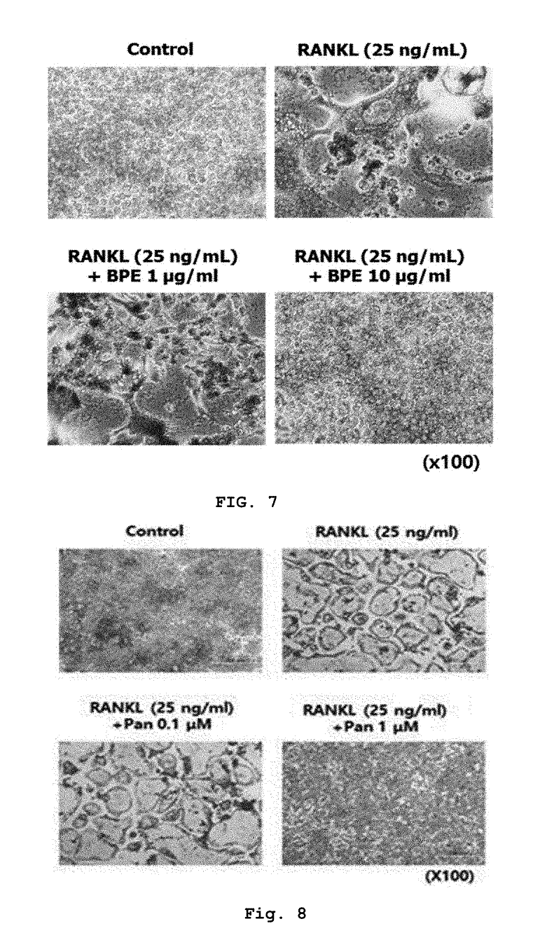

Using the same method as in Test example 1, RAW264.7 osteoclast precursor cells were attached in 6-well microtiter plates, and then α-MEM containing 10% bovine fetal serum was treated with 25 ng/ml RANKL (Peprotech) and panduratin A (0.1 μM and 1 μM) obtained in Example 2, respectively, thereby inducing differentiation for 4 days. The medium was exchanged every two days. After 4 days, supernatant was completely removed, and the cells were immobilized with 2% formaldehyde (Junsei chemical, Tokyo, Japan). Thereafter, TRAP staining was carried out using a leukocyte acid phosphatase kit (Sigma-Aldrich) according to manufacturer's guide line, and the osteoclast differentiation inhibitory effect of the Boesenbergia pandurata extract or panduratin A was analyzed through microscopic analysis. The results are shown FIG. 7 or 8.

As shown in FIG. 7 or 8, it could be confirmed that the osteoclast differentiation was inhibited by the treatment with the Boesenbergia pandurata extract or panduratin A. It could be especially confirmed that the differentiation was inhibited to a nearly similar level compared with a control in the Boesenbergia pandurata extract (10 μg/ml) treatment group or the panduratin A (1 μM) treatment group. These results indicate that the Boesenbergia pandurata extract or panduratin A of the present invention has excellent effect of inhibiting the bone resorption of osteoclasts.

Hereinafter, preparation examples of drugs and food containing, as an active ingredient, the Boesenbergiia panurata extract and/or panduratin A in Examples 1 and 2 according to the present invention will be described, but the present invention is not intended to be limited to these, which is for illustrative purposes. Drug and food compositions in preparation examples 1 and 2 were produced by conventional methods while the Boesenbergiia panurata extract and/or panduratin A having an excellent effect of preventing, ameliorating, or treating osteoporosis or alveolar bone loss were used according to the compositional ingredients and compositional ration thereof.

### Preparation Example 1: Drugs

### Preparation Example 1-1: Powder Preparation

A powder preparation was produced by mixing 50 mg of the Boesenbergiia panurata extract or panduratin A in Examples 1 and 2 with 2 g of crystalline cellulose and then charging the mixture in sealed packages through a conventional powder preparation producing method.

### Preparation Example 1-2: Tablet Preparation

A tablet preparation was produced by mixing 50 mg of the Boesenbergiia panurata extract or panduratin A in Examples 1 and 2, 400 mg of crystalline cellulose, and 5 mg of magnesium stearate, and then tableting the mixture through a conventional tablet preparation producing method.

### Preparation Example 1-3: Capsule Preparation

A tablet preparation was produced by mixing 30 mg of the Boesenbergiia panurata extract or panduratin A in Examples 1 and 2, 100 mg of whey protein, 400 mg of crystalline cellulose, and 6 mg of magnesium stearate, and then charging the mixture in gelatin capsules through a conventional capsule preparation producing method.

### Preparation Example 1-4: Injection Preparation

An injection preparation was produced by: dissolving active ingredients in distilled water for injection; adjusting the pH to about 7.5; mixing 100 mg of the panduratin A in Example 2, distilled water for injection, and a pH adjuster; charging the mixture in 2 ml-volume ampoules; and then sterilizing the ampoules.

### Preparation Example 2: Foods

Preparation Example 2-1: Preparation of Health Food

A health food was produced by mixing 1000 mg of the Boesenbergiia panurata extract or panduratin A in Examples 1 and 2, 70 ug of vitamin A acetate, 1.0 mg of vitamin E, 0.13 mg of vitamin B1, 0.15 mg of vitamin B2, 0.5 mg of vitamin B6, 0.2 ug of vitamin B12, 10 mg of vitamin C, 10 ug of biotin, 1.7 mg of nicotinamide, 50 ug of folic acid, 0.5 mg of pantothenic acid calcium, 1.75 mg of ferrous sulfate, 0.82 mg of zinc oxide, 25.3 mg of magnesium carbonate, 15 mg of potassium phosphate monobasic, 55 mg of dibasic calcium phosphate, 90 mg of potassium citrate, 100 mg of calcium carbonate, and 24.8 mg of magnesium chloride.

The mixing ratio may be arbitrarily changed. The above ingredients were mixed, and then granulated according to a conventional health food producing method, and the products may be used for the preparation of a health food composition according to a conventional method.

### Preparation Example 2-1: Preparation of Health Drink

A health drink was produced by adding purified water to 1000 mg of the Boesenbergiia panurata extract or panduratin A in Examples 1 and 2, 1000 mg of citric acid, 100 g of oligosaccharide, 2 g of a plum concentrate, 1 g of taurine to a total volume of 900 ml. The above ingredients were mixed, followed by heating and stirring at 85° C. for about 1 hour, and then the resultant solution was filtered, and taken in a 2 L-container, followed by sealing sterilization, and refrigeration, according to a conventional health drink producing method, and the products may be used for the preparation of a health drink composition.

### Preparation Example 2-3: Chewing Gum

Chewing gum was produced by mixing 20 wt % of a gum base, 76.9 wt % of sugar, 1 wt % of flavoring, 2 wt % of water, and 0.1 wt % of the Boesenbergiia panurata extract or panduratin A in Examples 1 and 2 according to a conventional method.

### Preparation Example 2-4: Candies

Candies were produced by mixing 60 wt % of sugar, 39.8 wt % of syrup, 1 wt % of flavoring, and 0.1 wt % of the Boesenbergiia panurata extract or panduratin A in Examples 1 and 2 according to a conventional method.

### Preparation Example 2-5: Biscuits

Biscuits were produced by mixing 25.59 wt % of first-grade weak flour, 22.22 wt % of first-grade medium flour, 4.80 wt % of refined sugar, 0.73 wt % of table salt, 0.78 wt % of glucose, 11.78% of palm shortening, 1.54 wt % of ammonium, 0.17 wt % of sodium bicarbonate, 0.16 wt % of sodium bisulfite, 1.45 wt % of rice flour, 0.0001 wt % of vitamin B, 0.04 wt % of milk flavoring, 20.6998 wt % of water, 1.16 wt % of dry milk powder, 0.29 wt % of substitute milk powder, 0.03 wt % of calcium phosphate, 0.29 wt % of spray salt, 7.27 wt % of spray milk, and the Boesenbergiia panurata extract or panduratin A in Examples 1 and 2 according to a conventional method.

### Preparation Example 3: Quasi-Drugs and Others

The following quasi-drugs were produced using the Boesenbergiia panurata extract or panduratin A prepared in the examples.

### Preparation Example 3-1: Toothpaste

Toothpaste containing the Boesenbergiia panurata extract or the panduratin derivative prepared in the examples, as shown in Table 1 below, was produced.

TABLE 1

| | | Preparation example | | | |
|---|---|---|---|---|---|
| | Ingredient name | 3-1-1 | 3-1-2 | 3-1-3 | 3-1-4 |
| Medicinal | Boesenbergiia | 0.1 | — | — | — |
| ingredient | panurata extract | | | | |
| | Panduratin A | — | 0.05 | — | — |
| | 4-Hydroxypanduratin A | — | — | 0.05 | — |
| | Isopanduratin A | — | — | — | 0.05 |
| Rubbing | Calcium carbonate | 40 | 40 | 40 | 40 |
| agent | | | | | |
| Wetting | Sorbitol liquid | 25.0 | 25.0 | 25.0 | 25.0 |
| agent | | | | | |
| Fluorine | Sodium | 0.1 | 0.1 | 0.1 | 0.1 |
| | monofluorophosphate | | | | |
| Foaming | Sodium lauryl | 2.0 | 2.0 | 2.0 | 2.0 |
| agent | sulfate | | | | |
| Binder | Carboxymethyl | 1.0 | 1.0 | 1.0 | 1.0 |
| | cellulose | | | | |
| Sweetening | Sodium saccharin | 0.2 | 0.2 | 0.2 | 0.2 |
| agent | | | | | |
| Preservative | Methyl paraben | 0.1 | 0.1 | 0.1 | 0.1 |
| Flavoring | Combined flavoring | 1.0 | 1.0 | 1.0 | 1.0 |
| agent | agent | | | | |
| Solvent | Purified water | Remainder | Remainder | Remainder | Remainder |

### Preparation Example 3-2: Gargling Liquid

The gargling liquid containing the Boesenbergiia panurata extract or the panduratin derivative prepared in the examples, as shown in Table 2 below, was produced.

TABLE 2

| | Preparation example | | | |
|---|---|---|---|---|
| Ingredient name | 3-2-1 | 3-2-2 | 3-2-3 | 3-2-4 |
| Boesenbergiia panurata | 0.1 | — | — | — |
| extract | | | | |
| Panduratin A | — | 0.05 | — | — |
| 4-Hydroxypanduratin A | — | — | 0.05 | — |
| Isopanduratin A | — | — | — | 0.05 |
| Glycerin | 5.0 | 5.0 | 5.0 | 5.0 |
| Poloxamer | 1.0 | 1.0 | 1.0 | 1.0 |
| Sodium fluoride | 0.1 | 0.1 | 0.1 | 0.1 |
| Sodium citrate | 0.2 | 0.2 | 0.2 | 0.2 |
| Sodium saccharine | 0.01 | 0.01 | 0.01 | 0.01 |
| Combined flavoring agent | 0.15 | 0.15 | 0.15 | 0.15 |
| Ethanol | 6.0 | 6.0 | 6.0 | 6.0 |
| Purified water | Remainder | Remainder | Remainder | Remainder |

### Preparation Example 3-3: Mouth Spray

Mouth spray containing the Boesenbergiia panurata extract or the panduratin derivative prepared in the examples, as shown in Table 3 below, was produced.

TABLE 3

| | Preparation example | | | |
|---|---|---|---|---|
| Ingredient name | 3-3-1 | 3-3-2 | 3-3-3 | 3-3-4 |
| Boesenbergiia panurata | 0.1 | — | — | — |
| extract | | | | |
| Panduratin A | — | 0.05 | — | — |
| 4-Hydroxypanduratin A | — | — | 0.05 | — |
| Isopanduratin A | — | — | — | 0.05 |
| Glycerin | 7.0 | 7.0 | 7.0 | 7.0 |
| Sodium citrate | 0.4 | 0.4 | 0.4 | 0.4 |
| Sodium fluoride | 0.02 | 0.02 | 0.02 | 0.02 |
| Sodium saccharine | 0.11 | 0.11 | 0.11 | 0.11 |
| Poloxamer | 1.0 | 1.0 | 1.0 | 1.0 |
| Methyl paraben | 0.1 | 0.1 | 0.1 | 0.1 |
| Propyl paraben | 0.05 | 0.05 | 0.05 | 0.05 |
| Combined flavoring agent | 0.12 | 0.12 | 0.12 | 0.12 |
| Ethanol | 60.0 | 60.0 | 60.0 | 60.0 |
| Purified water | Remainder | Remainder | Remainder | Remainder |

### Preparation Example 3-4: Edible Film

Edible films containing the Boesenbergiia panurata extract or the panduratin derivative prepared in the examples, as shown in Table 4 below, were produced.

TABLE 4

| | Preparation example | | | |
|---|---|---|---|---|
| Ingredient name | 3-4-1 | 3-4-2 | 3-4-3 | 3-4-4 |
| Boesenbergiia panurata | 0.1 | — | — | — |
| extract | | | | |
| Panduratin A | — | 0.05 | — | — |
| 4-Hydroxypanduratin A | — | — | 0.05 | — |
| Isopanduratin A | — | — | — | 0.05 |
| Flurane | 20.0 | 20.0 | 20.0 | 20.0 |
| Tween 80 | 0.1 | 0.1 | 0.1 | 0.1 |
| Carrageenann | 0.1 | 0.1 | 0.1 | 0.1 |
| Locust bean | 0.1 | 0.1 | 0.1 | 0.1 |
| Xanthan gum | 1.0 | 1.0 | 1.0 | 1.0 |
| Flavor | 1.0 | 1.0 | 1.0 | 1.0 |
| Puriifed water | Remainder | Remainder | Remainder | Remainder |

What is claimed is:

1. A method for ameliorating or treating a bone loss disease in a subject, the method comprising administering an effective amount of a composition comprising a Boesenbergia pandurata extract or a fraction thereof as an active ingredient to a subject in need thereof.

2. The method of claim 1, wherein the extract is an extract by at least one solvent selected from the group consisting of water, a C1-C6 organic solvent, subcritical water, and a supercritical fluid.

3. The method of claim 2, wherein the C1-C6 organic solvent is at least one selected from the group consisting of a C1-C6 alcohol, acetone, ether, benzene, chloroform, ethyl acetate, methylene chloride, hexane, cyclohexane, and petroleum ether.

4. The method of claim 1, wherein the fraction is a fraction of the Boesenbergia pandurata extract by a solvent containing at least one selected from the group consisting of hexane, chloroform, and ethyl acetate.

5. The method of claim 1, wherein the extract or fraction thereof comprises at least one panduratin derivative selected from the group consisting of compounds of Chemical Formulas 1 to 3:

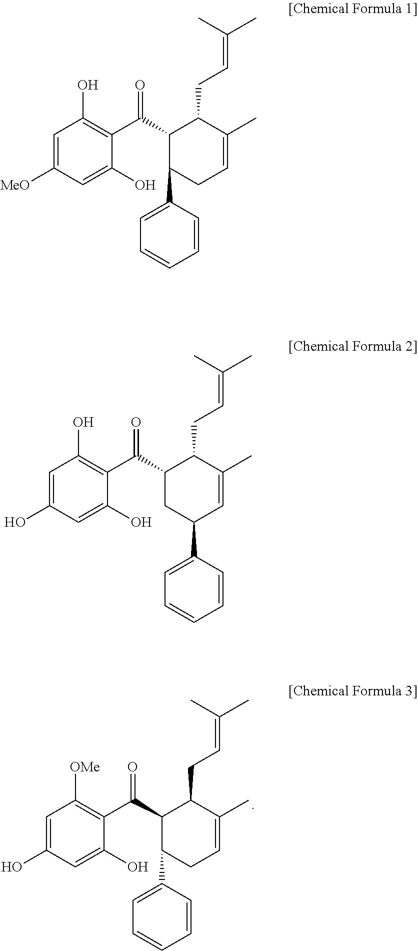 [SMILES: COc1cc(O)c(C(=O)[C@@H]2[C@H](CC=C(C)C)C(C)=CC[C@H]2c2ccccc2)c(O)c1 ; CC(C)=CC[C@@H]1C(C)=C[C@@H](c2ccccc2)C[C@@H]1C(=O)c1c(O)cc(O)cc1O ; COc1cc(O)cc(O)c1C(=O)[C@@H]1[C@@H](c2ccccc2)CC=C(C)[C@@H]1CC=C(C)C]

6. The method of claim 1, wherein the bone loss disease is selected from the group consisting of osteoporosis, Paget's disease, alveolar bone loss, osteomalacia, and osteodystrophy.

7. The method of claim 1, wherein the composition is a pharmaceutical composition, quasi-drug composition or food composition.

8. A method for ameliorating or treating a bone loss disease in a subject, the method comprising administering an effective amount of a composition comprising a panduratin derivative selected from the group consisting of compounds of Chemical Formulas 1 to 3 or a salt thereof as an active ingredient to a subject in need thereof:

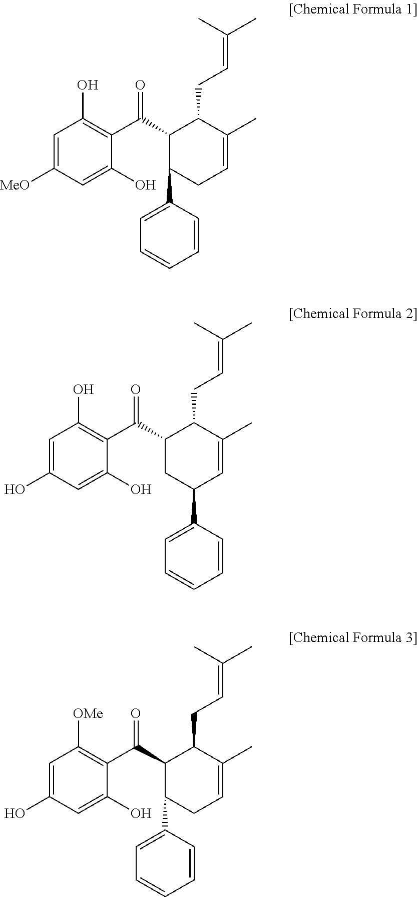 [SMILES: COc1cc(O)c(C(=O)[C@@H]2[C@H](CC=C(C)C)C(C)=CC[C@H]2c2ccccc2)c(O)c1 ; CC(C)=CC[C@@H]1C(C)=C[C@@H](c2ccccc2)C[C@@H]1C(=O)c1c(O)cc(O)cc1O ; COc1cc(O)cc(O)c1C(=O)[C@@H]1[C@@H](c2ccccc2)CC=C(C)[C@@H]1CC=C(C)C]

9. The method of claim 8, wherein the panduratin derivative is extracted from Boesenbergia pandurata.

10. The method of claim 8, wherein the bone loss disease is selected from the group consisting of osteoporosis, Paget's disease, alveolar bone loss, osteomalacia, and osteodystrophy.

11. The method of claim 8, wherein the composition is a pharmaceutical composition, quasi-drug composition or food composition.
